(19) United States
(12) Patent Application Publication
(10) Pub. No.: US 2011/0034821 A1
(43) Pub. Date: Feb. 10, 2011

(54) Increasing the information transfer rate of brain-computer interfaces

(76) Inventor: Frank Edughom Ekpar, Aizuwakamatsu City (JP)

Correspondence Address: Frank Edughom Ekpar, Matsunaga 1-17-26-A107, Ikki-Machi, Aizuwakamatsu City, Fukushima, 965-0001, JP

(21) Appl. No.: 12/462,671

(22) Filed: Aug. 10, 2009

(51) Int. Cl. A61B 5/0478; G06G 7/60

(52) U.S. Cl. 600/544; 703/11

(57) ABSTRACT

Methods of increasing the rate of information transfer in brain-computer interface systems are disclosed. The present invention also discloses methods, devices and systems for the navigation of information representing neuronal or brain activity and the extraction of useful and/or actionable data from such information.

### CROSS-REFERENCE TO RELATED APPLICATIONS

This U.S. Non-Provisional Application claims the benefit of U.S. Provisional Application Ser. No. 61/137,891, file on Aug. 5, 2008, herein incorporated by reference.

### BACKGROUND OF THE INVENTION

1. Field of the Invention

The present invention relates generally to the fields of neuro-informatics, bio-informatics, bio-engineering, and allied fields. In particular, the invention relates to methods of increasing the information transfer rate (measured in bits per second: the product of information transfer per presentation—in bits per item—and the presentation rate—in items per second) of brain-computer interface systems.

2. Description of the Prior Art

Brain-computer interfaces (BCI) are systems that serve as communication pathways between humans (and generally animals) and machines. In BCIs, signals corresponding directly or indirectly to physiological and cognitive processes in the subject could be translated into commands that could be used to control external devices. Conversely, signals from external sensors could be transformed into a suitable format and used to induce perceptions in the subject that would ordinarily be induced through the normal operation of the body's natural sensory organs. Thus, BCIs provide means of circumventing the usual motor-sensory pathways in the subject and could be harnessed as an independent channel of communication with the subject's environment. For subjects with impairments, the circumvention of the traditional motor-sensory pathways facilitated by BCIs hold the promise of a viable means of restoring interaction with the environment that would otherwise be impossible or difficult to attain. Healthy subjects could also use BCIs as alternative and potentially more intuitive communication channels.

A variety of methods and devices—each with its own set of advantages and drawbacks—can be used to acquire brainwave data. These generally fall into two broad categories—invasive and non-invasive. Invasive methods and systems are characterized by the utilization of intra-cranial means of recording signals while non-invasive methods and systems typically involve the measurement of signals without direct contact with the cells generating the signals. The electrocorticography (ECoG) technique described by Leuthardt; Eric C. et al. in U.S. Pat. No. 7,120,486 involves the recording of the electrical activity of the cerebral cortex by means of electrodes placed directly on it, either under the dura mater (subdural) or over the dura mater (epidural) but beneath the skull and is thus an example of an invasive method of brainwave signal acquisition. Systems based on functional magnetic resonance imaging (fMRI), positron emission tomography (PET), single photon emission computerized tomography (SPECT), electroencephalography (EEG), magnetoencephalography (MEG), and functional near-infrared spectroscopy (fNIRS) provide non-invasive means of brainwave recording and depend on a variety of principles ranging from neurovascular coupling (the relationship between blood flow in neural cell populations and cognitive activity involving the participation of said neural cell populations) to electrophysiological analyses. Invasive techniques generally provide more accurate representations of neuronal activity but are hampered by the associated risks and inconvenience of brain surgery (for implantation of the recording device) and degeneration of signal quality due to encapsulation of the recording electrodes by fibrous tissue and/or destruction of neighboring cells by the electrodes.

Currently, the majority of non-invasive BCIs are based on the well known. electroencephalography (EEG) technique owing to its relative portability, low cost, high temporal resolution and ease of operation. Examples of BCIs based on EEG and/or other non-invasive recordings include those disclosed in U.S. Pat. No. 5,638,826, U.S. Pat. No. 7,403,815 and U.S. Pat. No. 6,349,231. The spatial resolution of contemporary EEG-based BCIs is quite low—with systems typically comprising between 1 and 256 electrodes, each of which aggregates signals from massive neuronal populations. Furthermore, the signals are heavily attenuated on their journey through the skull and are thus susceptible to corruption by noise from other signal-emitting physiological processes in the subject and disturbances from the environment.

Techniques, algorithms and systems that remedy the shortcomings of EEG-based BCIs are well known and widely reported in the literature. Writing in the Proceedings of the United States National Academy of Sciences (2004 Dec. 21; 101(51): 17849-17854), Jonathan R. Wolpaw and Dennis J. McFarland describe an adaptive algorithm that uses a simple linear combination of relevant features to improve the effectiveness of a non-invasive BCI designed for 2-dimensional computer cursor control. Although the method described by Jonathan R. Wolpaw et al. provides better results than some competing methods by adapting the features selected for classification to the specific features that the user is best able to control, it is still hampered by the major drawback of high sensitivity to individual brainwave characteristics and the requirement for long training periods. The information transfer rate of EEG-based BCIs is currently in the range of 5 to 25 bits per second which is too low to permit widespread use of such BCIs in practical applications.

### SUMMARY OF THE INVENTION

It is an object of the present invention to overcome the limitations of the prior art set forth above by providing a method for increasing the information transfer rate of brain-computer interfaces. Another object of the present invention is to provide means of navigating information representing the state and/or activities of neural populations or related entities or simulations of same. It is also an object of the present invention to provide means of extracting useful and/or actionable information from representations and/or navigation of information representing the state and/or activities of neural populations or related entities or simulations of same.

### BRIEF DESCRIPTION OF THE DRAWINGS

FIG. 1 illustrates the preferred embodiment of the present invention.

### DETAILED DESCRIPTION OF THE PREFERRED EMBODIMENTS

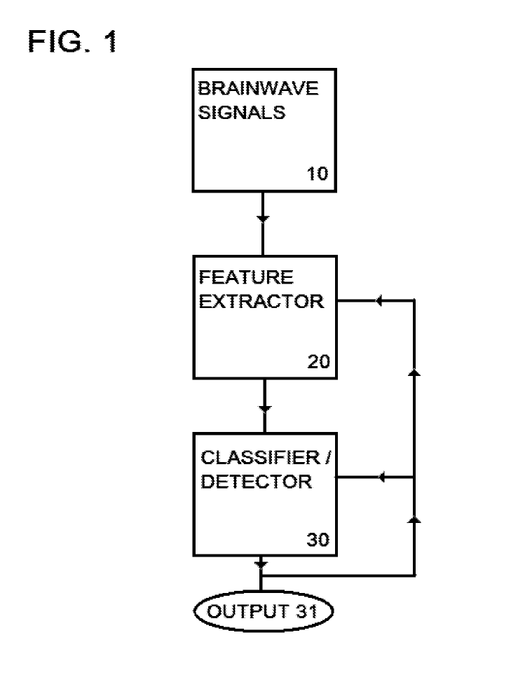

In FIG. 1, an illustration of the preferred embodiment of the present invention, brainwave signals corresponding to a subject's physiological or event-related cognitive state are acquired by the brainwave acquisition unit, 10. A suitable recording device based on electroencephalograph, electrocorticograph, near-infrared spectrograph, etc, could be used as the source of the brainwave signals from the subject. The spatial and temporal resolution of contemporary brainwave recording equipment is limited. Using an ultra-dense sensor network (possibly comprising nano-probes/nano-electrodes) capable of recording the activity (electrical, electromagnetic, etc) of individual neurons or neural populations consisting of a relatively small number of neurons (in the order of 1 to 100 neurons per population), more accurate brainwave readings could be obtained. The vast number of data points acquired from such a dense sensor network poses serious processing challenges.

Numerous studies have shown that it is valid to consider information processing in human (and other animal) brains as a hierarchical and distributed model in which information representing stimuli or physiological states could be decomposed into simpler units of information and the processing of these simpler units distributed among different neural populations. The present invention adopts this approach to the processing of brainwave signals. Accordingly, the feature extraction unit—depicted generally as 20 in FIG. 1—extracts representations of salient features from the incoming brainwave signals. The exact features selected and how these are represented depends on the application. For a given classification task, a set of salient features is selected by a separate feature extraction unit. Each feature extraction unit is coupled to a classification/detection unit, 30, that is trained to recognize/detect that specific feature. The classification units preferably classify/detect features in parallel. With the decreasing cost of multi-core computers and refinements in parallel programming languages and systems, this scheme could be amenable to straightforward implementation on general-purpose consumer personal computers. In the absence of multi-core hardware, multi-threaded programming could be used to implement parallel feature processing. The output of the classifier/detector, labeled 31 in FIG. 1, is fed back to the feature extractor, 20 and classifier, 30 and used to adaptively modify the behavior of the feature selector and/or classifier with a view to providing more accurate feature selection and/or classification. This processing is repeated (preferably in parallel) for each feature at each stage of the hierarchy with the classification results from all salient features for each target class recombined to generate the final output which in turn could be used to control external devices. Jonathan R. Wolpaw and Dennis J. McFarland describe an adaptive algorithm that uses a simple linear combination of relevant features to improve the effectiveness of a non-invasive BCI designed for 2-dimensional computer cursor control in United States National Academy of Sciences (2004 Dec. 21; 101(51): 17849-17854). The method described by Wolpaw et al. is limited by the requirement for extensive training of the user. In contrast, the present invention is directed towards a method that uses the hierarchical decomposition of the feature space to provide a means of identifying and adaptively modifying/classifying simpler features (that are more likely to have characteristics common to most subjects) in parallel which are then re-combined to generate the final output thus obviating or at least mitigating the need for extensive subject training. This increases the information transfer rate (simpler features can be classified faster and more accurately in parallel using simpler algorithms) and expands the scope of practical applications of BCIs.

For ultra-dense sensor arrays, the massive amounts of data generated could be dealt with using the dynamic view prediction method described in co-pending U.S. provisional patent application No. 60/965,715—by the present inventor. In this case, the target “view” would represent the subset of the entire data set that can be processed or viewed (the signal at each sensor locus could be viewed as the color of a pixel in an image in which sensor loci are viewed as pixels) at any given time using the resources of the available processing/rendering system. Suitable embodiments of the versatile imaging device described in U.S. Pat. No. 7,567,274 by the present inventor could also be used to acquire signals from neuronal or brain activity and/or to navigate or view representations of the information.

Navigation of the data extracted from signals representing the states and/or activities of neurons or related entities could provide insights into the underlying physiological and/or other processes and conditions. Such insights could inform diagnosis and/or treatment of abnormal conditions and/or confirmation of normal operation.

The methods, systems and devices described herein need not be limited to biological neurons or similar entities but can be applied to simulations of such entities. Such simulations could consist of computer programs implementing models of characteristics of the biological or similar entities they represent.

Furthermore, the sensors or probes used to decipher the state, activities and other relevant characteristics of the neurons or similar entities could be simulated. As is the case with the subject entities themselves, such simulations could be implemented as computer programs that model the relevant characteristics and/or behavior of the sensors or probes.

It should be understood that numerous alternative embodiments and equivalents of the invention described herein may be employed in practicing the invention and that such alternative embodiments and equivalents fall within the scope of the present invention.

What is claimed is:

1. An apparatus for extracting information from neurons or similar entities or simulation of same, said apparatus comprising one or more sensor elements responsive to signals from said neurons or similar entities and transforming said signals into one or more representative formats.

2. The apparatus recited in claim 1 wherein said one or more sensor elements is adapted to generate signals corresponding to the state of said neurons or similar entities or simulation of same.

3. The sensor elements recited in claim 2 wherein said signal corresponding to said state of said one or more neurons or similar entities or simulation of same is electromagnetic.

4. The sensor elements recited in claim 2 wherein said signal corresponding to said state of said one or more neurons or similar entities or simulation of same is acoustic or ultrasonic.

5. An apparatus for extracting one or more representations of salient features underlying the activities or states of neurons or similar entities or simulation of same.

6. An apparatus adapted to perform hierarchical decomposition of the feature space of features extracted from one or more representations of the activities or states of neurons or similar entities or simulation of same to provide a means of identifying and adaptively modifying/classifying simpler features (that are more likely to have characteristics common to most subjects) in parallel which are then re-combined to generate a signal or set of signals thus obviating or at least mitigating the need for extensive subject training.

7. A method of extracting information from neurons or similar entities or simulation of same, said method using input from one or more sensor elements or simulations thereof responsive to signals from said neurons or similar entities or simulation of same and transforming said signals into one or more representative formats.

8. The method recited in claim 7 wherein said one or more sensor elements or simulation thereof is adapted to generate signals corresponding to the state of said neurons or similar entities or simulation of same.

9. The sensor elements recited in claim 8 wherein said signal corresponding to said state of said one or more neurons or similar entities or simulation of same is electromagnetic.

10. The sensor elements recited in claim 8 wherein said signal corresponding to said state of said one or more neurons or similar entities or simulation of same is acoustic or ultrasonic.

11. A method of extracting one or more representations of salient features underlying the activities or states of neurons or similar entities or simulation of same.

12. A method of performing hierarchical decomposition of the feature space of features extracted from one or more representations of the activities or states of neurons or similar entities or simulation of same to provide a means of identifying and adaptively modifying/classifying simpler features (that are more likely to have characteristics common to most subjects) in parallel which are then re-combined to generate a signal or set of signals thus obviating or at least mitigating the need for extensive subject training.
